(19) United States
(12) Patent Application Publication
(10) Pub. No.: US 2024/0001106 A1
(43) Pub. Date: Jan. 4, 2024

(54) ALIGNMENT GARMENT FOR USE WITH A FULLY IMPLANTABLE SYSTEM

(71) Applicant: Medtronic, Inc., Minneapolis, MN (US)

(72) Inventors: Stephen M. Nelson, Plymouth, MN (US); David J. Peichel, Minneapolis, MN (US); Eric A. Schilling, Ham Lake, MN (US); Nathanael O. Mason, Holliston, MA (US); Robert Scadding Moir, Cambridge (GB); Lillian E. Clarke, Cambridge (GB); Ismail Akram, Cambridge (GB); Glenn Le Faou, Hertfordshire (GB); Richard James Andrews, Cambridge (GB); Jordi Vilanova Hanusaik, Cambridge (GB); Anna Rebecca Louise Wilson, Cambridge (GB); Arron Carmo do Rosario Rodrigues, Cambridge (GB)

(21) Appl. No.: 18/251,328

(22) PCT Filed: Sep. 21, 2021
(86) PCT No.: PCT/US2021/051200

§ 371 Date: May 1, 2023

Related U.S. Application Data
(60) Provisional application No. 63/108,618, filed on Nov. 2, 2020.

(51) Int. Cl. A61M 60/875

(52) U.S. Cl. CPC A61M 60/875; A61M 2209/088

(57) ABSTRACT

An alignment garment for holding an external coil in a predetermined location. The alignment garment may comprise an attachment mechanism for holding the external coil in the predetermined location. The alignment garment may also have a plurality of straps with a first strap in the plurality of straps being secured to the attachment mechanism and a second strap of the plurality of straps being secured to the attachment mechanism.

### FIELD

The present technology is generally related to an alignment system for use with an implantable medical device system that has the transfer of energy from a power source outside the body to an implanted medical device inside the body.

### BACKGROUND

Mechanical circulatory support devices, including ventricular assist devices (“VADs”), are used for people that have weakened hearts and/or heart failure. VADs can aid the heart's natural pumping to increase the amount of blood flow throughout the body. VADs require various external and internal components to work together to run the system so that blood is received from the patient's heart and the VAD pumps the blood back into arterial circulation. When the external and internal components of the VAD work together properly, an individual with a weakened heart and/or advanced heart failure can lead a more active and fuller life.

A transcutaneous energy transfer system (“TETS”) may be used to power implantable devices such as the VADs. Generally, a TETS can transfer energy from an external transmission coil to a receiving coil that is implanted under the skin. A TETS may be used to supplement, replace, or charge an implanted power source such as a rechargeable battery.

Some of the components that are used in the VADs include an external transmission coil and an implanted receiving coil. Various components must be properly aligned to prevent an interruption of the operation of the VADs. Movements of a patient can cause the misalignment of components from the VADs and/or the TETS which may cause the system to malfunction and in certain instances fail when power is not being transferred into the system.

### SUMMARY

The techniques of this disclosure generally relate to an alignment system for a wireless energy transfer system, including wearable medical devices that use a transcutaneous energy transfer system (“TETS”), that require the alignment of certain external and internal components to continually facilitate the effective transfer of energy.

In one aspect, an alignment garment for holding an external coil in a predetermined location, the alignment garment comprising: an attachment mechanism for holding the external coil in the predetermined location; a plurality of straps including a first strap of the plurality of straps being secured to the attachment mechanism and a second strap of the plurality of straps being secured to the attachment mechanism.

In another aspect, each strap in the plurality of straps are adjustable to different lengths.

In another aspect, the first strap of the plurality of straps has a first portion and a second portion, the first portion being configured to be secured to the attachment mechanism and the second portion being secured to the second strap of the plurality of straps.

In another aspect, the first strap of the plurality of straps is between 10 and 30 inches.

In another aspect, the second portion of the first strap of the plurality of straps is made from a material that has a low elasticity to limit movement in the second portion of the strap.

In another aspect, the first portion of the first strap of the plurality of straps is moveable along a single axis.

In another aspect, the second strap of the plurality of straps has a first portion and a second portion, the first portion and the second portion being configured to be secured to the attachment mechanism.

In another aspect, each strap of the plurality of straps includes at least one slider, the at least one slider being configured to adjust each strap of the plurality of straps to different lengths.

In another aspect, the at least one slider is made from a non-conductive material.

In one aspect, an alignment garment for holding an external coil in a predetermined location, the alignment garment comprising: an attachment mechanism for holding the external coil in the predetermined location; a first strap having a circumference of between 20 and 60 inches; a second strap secured to the attachment mechanism and the first strap, the second strap having a length of between 15 and 40 inches; a third strap secured to the attachment mechanism and secured to the first strap; and a fourth strap secured to the attachment mechanism and the first strap.

In another aspect, the third strap has a length of between 5 and 15 inches.

In another aspect, the fourth strap has a length of between 5 and 15 inches.

In another aspect, the first strap has a first slider proximate where the third strap is secured to the first strap and the first strap has a second slider proximate where the fourth strap is secured to the first strap.

In another aspect, the first strap has at least three sliders adjust the length of the first strap.

In another aspect, the second strap is made from a material that has a low elasticity to limit the movement of the second strap.

In another aspect, the first strap has a securing mechanism configured to releasably secure the first strap.

In another aspect, the first strap has a reinforcing mechanism disposed where the first strap is secured to the second strap.

In one aspect, an alignment garment for holding an external coil in a predetermined location, the alignment garment comprising: an attachment mechanism for holding the external coil in the predetermined location; a first strap having a circumference of between 20 and 60 inches; a second strap secured to the attachment mechanism and the first strap, the second strap having a length of between 15 and 40 inches; a third strap secured to the attachment mechanism and secured to the first strap, the third strap having a length of between 5 and 15 inches; and a fourth strap secured to the attachment mechanism and the first strap, the fourth strap having a length of between 5 and 15 inches.

In another aspect, the first strap has a first slider proximate where the third strap is secured to the first strap and the first strap has a second slider proximate where the fourth strap is secured to the first strap.

In another aspect, the second strap is made from a material that has a low elasticity to limit the movement of the second strap.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front inside of the body view of a patient with a left ventricular assist device, receiving coil, and controller fully implanted within the patient;

FIG. 2 is a front outside view of the body view of the patient shown in FIG. 1 showing a battery and transmission coil coupled to the patient;

FIG. 3 is a front view of the receiving coil and controller shown in FIG. 1;

FIG. 4 is a front view of an exemplary embodiment of the alignment system for the lateral region of the patient's torso;

FIG. 5 is a rear view of the exemplary embodiment in FIG. 4;

FIG. 6 is a front view of the exemplary embodiment in FIG. 4 on the body of the patient;

FIG. 7 is a rear view of the exemplary embodiment in FIG. 4 on the body of the patient;

FIG. 8 is a front view of an exemplary embodiment of the alignment system for the pectoral region of the patient's body;

FIG. 9 is a rear view of the exemplary embodiment in FIG. 12;

FIG. 10 is a front view of the exemplary embodiment in FIG. 12 on the body of the patient; and

FIG. 11 is a rear view of the exemplary embodiment in FIG. 12 on the body of the patient.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

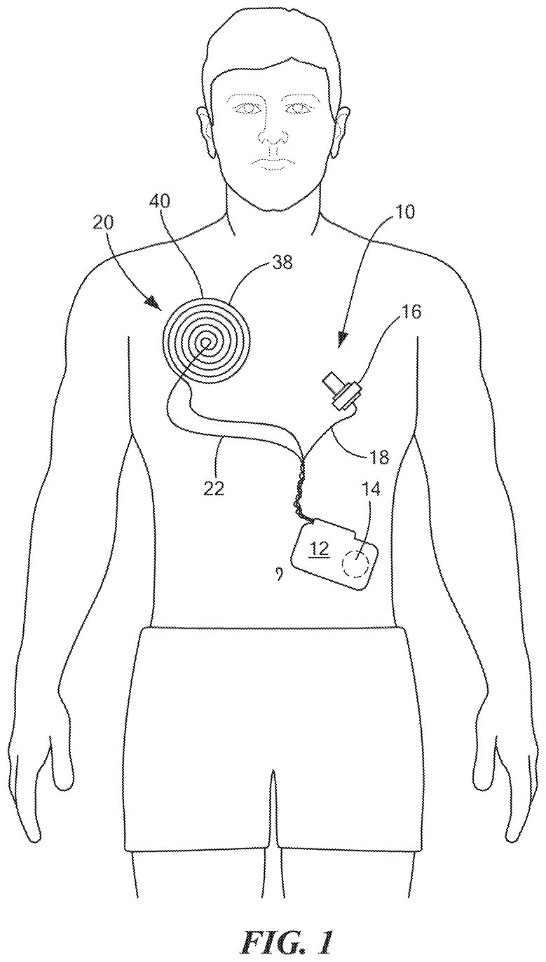

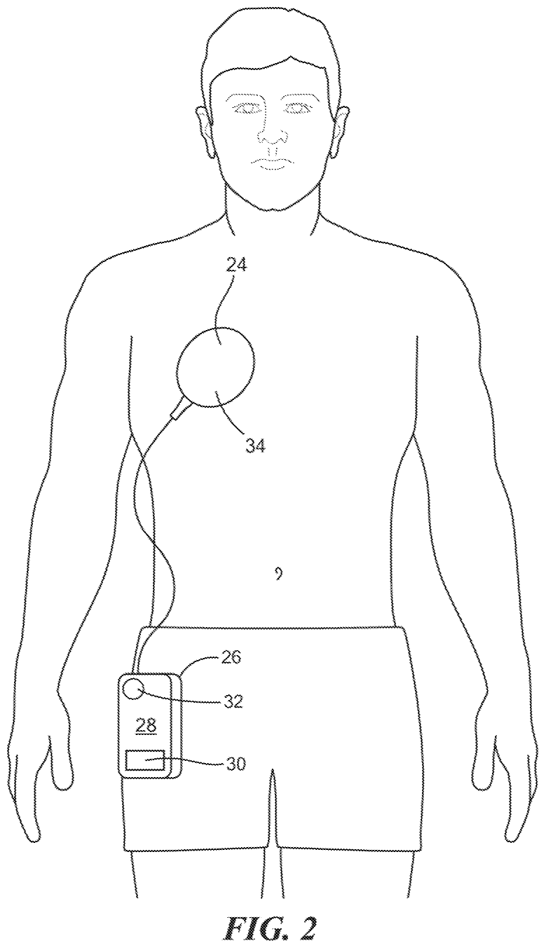

Referring now to the drawings in which like reference designators refer to like elements there is shown in FIGS. 1 and 2 an exemplary transcutaneous energy transfer system (“TETS”) constructed in accordance with the principles of the present application and designated generally as “10.” While there is discussion about a particular TETS, it will be understood that the TETS can be used for wireless energy transfer for another type of implantable medical device and can be in a variety of different configurations. The TETS 10 may be fully implantable within a patient, whether human or animal, which is to say there are no percutaneous connections between the implanted components of the TETS 10 and the components outside of the body of the patient. In the configuration shown in FIG. 1, the TETS 10 includes an internal controller 12 implanted within the body of the patient. The internal controller 12 may include a power source 14 configured to power the components of the controller and provide power to one or more implantable medical devices, for example, an implantable blood pump such as a ventricular assist device system (“VADs”) 16 implanted within the left ventricle of the patient's heart. The power source 14 may include a variety of different types of power sources including an implantable battery. The VADs 16 may include centrifugal pumps, axial pumps, or other kinds electromagnetic pumps configured to pump blood from the heart to blood vessels to circulate around the body. One such centrifugal pump is the HVAD and is shown and described in U.S. Pat. No. 7,997,854, the entirety of which is incorporated by reference. One such axial pump is the MVAD and is shown and described in U.S. Pat. No. 8,419,609. In an exemplary configuration, the VADs 16 is electrically coupled to the internal controller 12 by one or more implanted conductors 18 configured to provide power to the VADs 16, relay one or more measured feedback signals from the VADs 16, and/or provide operating instructions to the VADs 16.

Continuing to refer to FIG. 1, a receiving coil 20 may also be coupled to the internal controller 12 by, for example, one or more implanted conductors 22. In an exemplary configuration, the receiving coil 20 may be implanted subcutaneously proximate the thoracic cavity, although any subcutaneous position may be utilized for implanting the receiving coil 20. The receiving coil 20 is configured to be inductively powered through the patient's skin by a transmission coil 24 (seen in FIG. 2) disposed opposite the receiving coil 20 on the outside/exterior of the patient's body. For example, as shown in FIG. 2, a transmission coil 24 may be coupled to an external controller 26 having a power source 28, for example, a portable battery carried by the patient. The transmission coil 24 may have raised portions or be a smooth surface. The raised portions may be configured to engage with different components of the alignment system. In one configuration, the battery is configured to generate a radiofrequency signal for transmission of energy from the transmission coil 24 to the receiving coil 20. The receiving coil 20 may be configured for transcutaneous inductive communication with the transmission coil 24.

The external controller 26 may further include a display 30 configured to provide information to the patient in one or more ways including audibly, visually, and/or tacitly. Additionally, the external controller 26 may further include processing circuitry 32 which can process and transmit information. The processing circuitry 32 may perform various simple and complex operations including, but not limited to, the amplification of signals, computations, and the movement of data from one place to another. The various simple and complex operations may be performed by the processing circuitry 32 in the external controller 26. The processing circuitry 32 may include one or more processors and memory. The external controller 26 may further comprise software, which is stored in, for example, memory at the external controller 26, or stored in external memory (e.g., database, storage array, network storage device, etc.) accessible by the external controller 26. The software may be executable by the processing circuitry 32. The software may include a client application operable to provide a service to a human or non-human user via the external controller 26, with the support of a host computer.

Referring back to FIG. 2, the transmission coil 24 is optionally housed within sealed packaging 34 to protect the transmission coil 24 and is optionally secured by a garment 36, that is configured to maintain the transmission coil 24 in a fixed position for power transmission to the receiving coil 20.

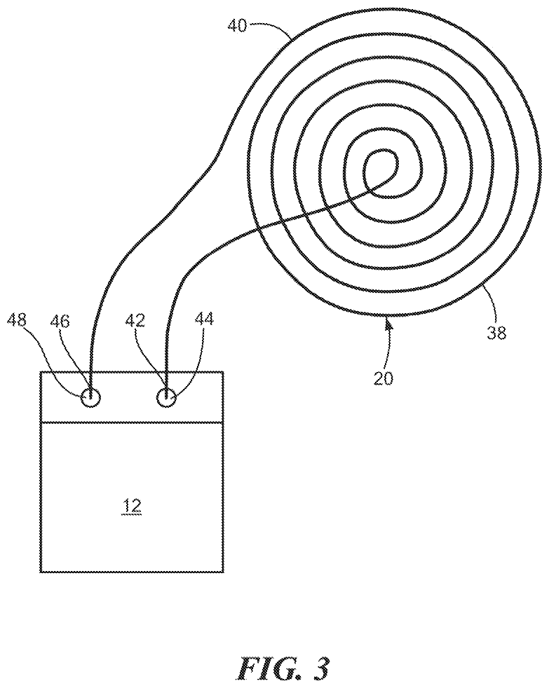

Referring now to FIGS. 1 and 3, the receiving coil 20 includes at least one conductor 38 defining a coil 40 and configured to power the VAD 16. The receiving coil 20 may be any shape or size and, for example, in one configuration, the receiving coil 20 may define a substantially planar coil defining a diameter such that is substantially co-planar with an interior surface of the dermis. The receiving coil 20 may be coupled with the internal controller 12 with a first end 42 being electrically coupled to a first coupling 44 of the internal controller 12 and a second end 46 being coupled to a second coupling 48 of the internal controller 12 such that a voltage may be applied to the receiving coil 20. Alternatively, the receiving coil 20 may be coupled with the internal controller 12 using joints or any other configuration. Although not shown, temperature sensors may be found throughout the TETS 10.

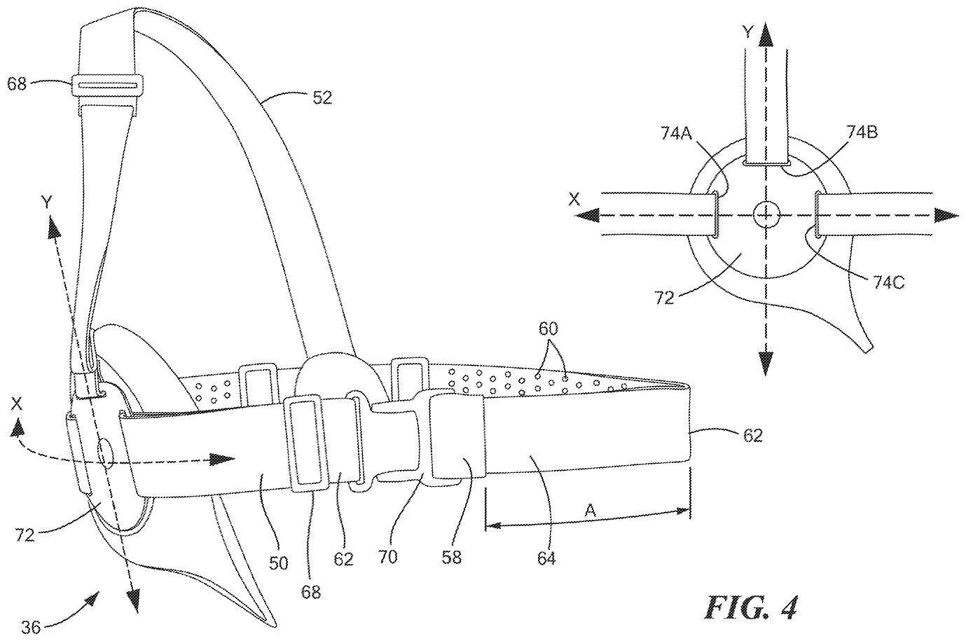

Referring to FIGS. 4-7 wherein an exemplary garment 36 is shown. The garment 36 may be used to help maintain the position and alignment of certain external and internal components of the wireless energy transfer system to allow for the transmission of energy between the external and internal components to help maintain the charge and energy of the internal components. As a non-limiting example as shown in FIG. 1, the implanted charging component may be the receiving coil 20. It will be understood that the implanted charging component may be any other implanted component but will be generally referred to in the application as the receiving coil 20. Also, the external charging component may be any external charging component, but as a non-limiting example as shown in FIG. 2, the external charging component may be the transmission coil 24. It will be understood that the external charging component may be any other external component but will be generally referred to the in application as the transmission coil 24. To allow for energy to transfer in the TETS, the transmission coil 24 and the receiving coil 20 must be properly aligned.

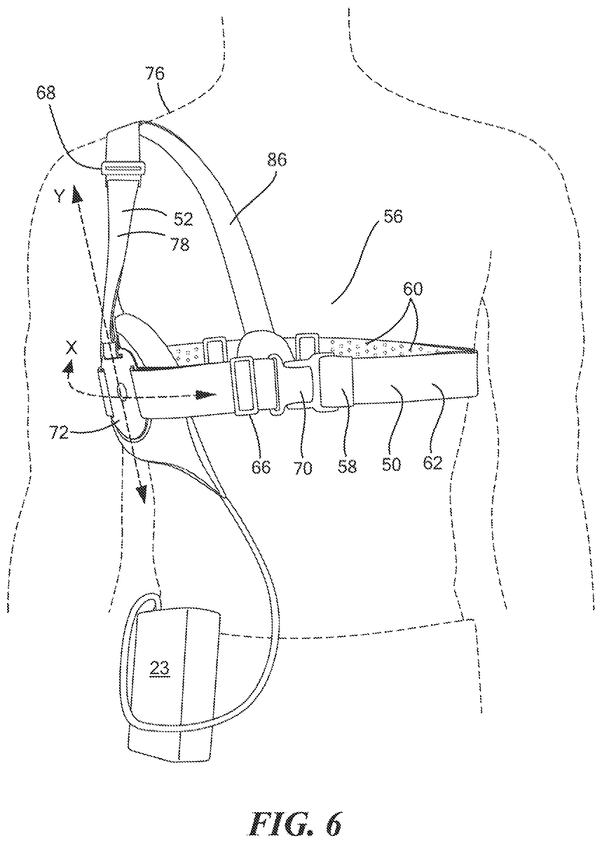

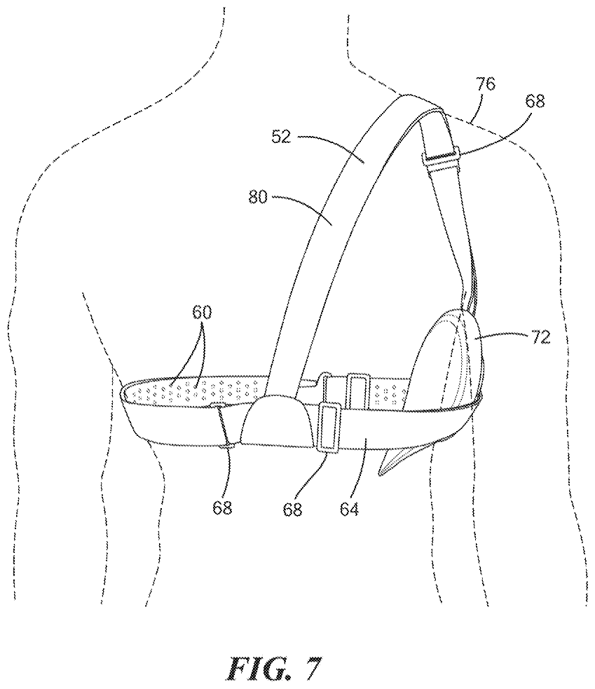

Continuing to refer to FIGS. 4-7, the garment 36 may be used to maintain the alignment of the external charging component/transmission coil 24 over the implanted charging component/receiving coil 20 within, for example, a 2 inch tolerance along the planar and linear axes of the components for the TETS system so that power can continually be transferred effectively and efficiently. In one embodiment, the garment 36 may include a first strap 50 and a second strap 52. The first strap 50 may be sized to fit around the torso 56 of the patient, as shown, for example in FIGS. 6 and 7. The first strap 50 may be made from a flexible material so that the first strap 50 may be configured to fit a variety of different body shapes and sizes. The first strap 50 may be made from one continuous portion and/or segment and/or the first strap may be made from various portions and/or segments. In one configuration, the first strap 50 may include a first portion 58 which may be the entire first strap 50, only a segment of the first strap 50, or more than one segment of the first strap 50. In the embodiment in FIG. 4, the first portion 58 of the first strap 50 is one segment of the first strap. The first strap 50, or at least one segment of the first strap 50, may be made from a flexible and stretchy material, including a plush elastic, to make the first strap 50 adjustable and able to fit around different sizes and shapes. The plush elastic may have an elastic foundation and have one soft and velvety side and a second side that is not as soft. In one example, the first strap 50 may have a first side 60 and a second side 62 opposite the first side 60. The first side 60 may be configured to contour around the skin of the torso 56 and be made from the soft and velvety material as the first side 60 may be in direct contact with the skin and the second side 62 may be opposite the first side and not in direct contact with the patient's skin.

Continuing to refer to FIG. 4, the first strap 50 may also include a second portion 64 which may be only a segment of the first strap 50, or more than one segment of the first strap 50. The second portion 64 may be made from a material that has a stretchy and elastic quality while also being able to stay in place on the patient's body. For example, the second portion 64 of the first strap 50 may be made out a material which is stretchy on the second side 62 and has a material on the first side 60 that is designed to keep the second portion 64 in place on the patient's skin. An example of material that may be used on the second portion 64 may be a dot silicon elastic which is stretchy on the second side 62 and has silicon dots on the first side 60 to help keep the first strap 50 in place.

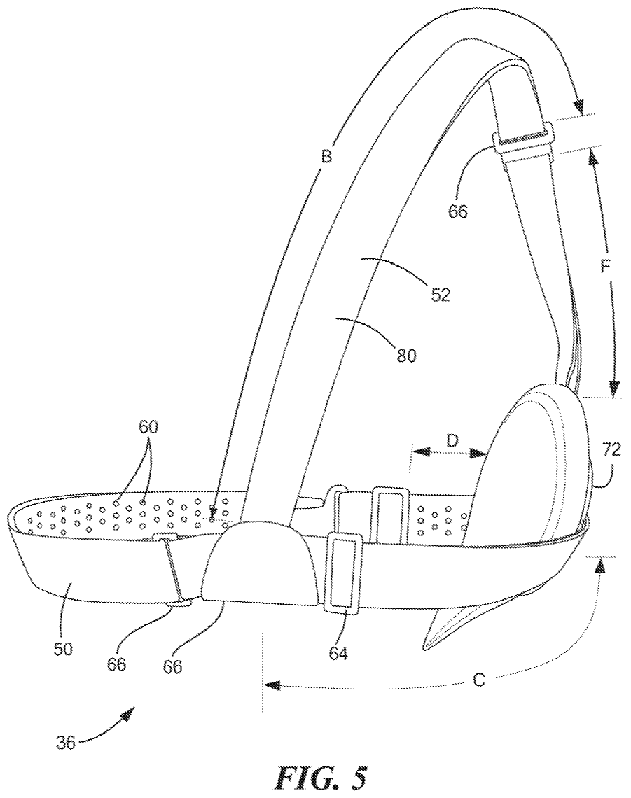

In FIG. 4, the first strap 50 may also include a first material segment 66 (shown in FIG. 5) and the first material segment 66 may be configured so that first strap 50 may comfortably fit around a portion of the torso 56 of the patient. The first material segment 66 may be made out of a fabric that be placed in direct contact with a patient's skin on the first side 60 of the first strap 50 and cause minimal irritation including fabrics like polyester, viscose, rayon, and spandex and/or a combination of these fabrics. In one example, the material may be a double knit jersey dress fabric. The first strap 50 and the second strap 52 may also include at least one slider 68 and/or more than one slider 68. It will be understood that reference to the slider 68 can be one slider 68 or more than one slider 68. The sliders 68 may be configured to lock in place through friction created with any portion of the garment 36. The number of sliders 68 on the first strap 50 may be customized for the patient so that the first strap 50 can easily and comfortably fit around the torso 56 of the patient. The at least one slider 68, for example, may be approximately 1.5 inch plastic slider, a 1.2 inch plastic slider, a 1.2 inch plastic slider/loop, or any other size and shape slider 68 depending upon the size, shape, and thickness of the first strap 50 as well as the second strap 52 and the size and shape of the patient. The slider 68 may be made out of any material to make it comfortable for the patient to wear for prolonged periods of time, be large enough to be sturdy when secured to the first strap 50, and any portion of the slider 68 that is in direct contact with the patient's skin may be covered with a material and/or fabric to make it comfortable to wear for long periods of time. The slider 68 may be made from non-conductive material including plastics or other non-conductive material. Alternatively, the sliders 68 may also be made from conductive materials including metal and/or another conductive material. The placement of the slider 68 on the garment 36 may determine if a conductive material or non-conductive material is to be used as certain conductive materials may interfere with the energy transfer and/or cause harm to the patient.

Continuing to refer to FIG. 4, the first strap 50 may also include a first securing mechanism 70 to make it easier for a patient to secure the first strap 50 around the torso 56. In one example, the first securing mechanism 70 may be a buckle such as a frog clip or another type of releasable clip to help secure the first strap 50 in place and make it easy to release the first strap 50 from being secured around the torso 56 of the body. The first securing mechanism 70 may provide a single point of connection that is releasable to allow an individual to secure the garment 36 in place on the body. Alternatively, the first securing mechanism 70 may have more than one point of connection that is releasable to allow an individual to secure the garment 36 in place on the body. The use of the first securing mechanism 70 may allow the patient to easily open the first strap 50 and secure the first strap 50 around the torso 56. The first securing mechanism 70 can be used to tighten and loosen the first strap 50 as well. In a non-limiting example, the first material segment 66 may be secured to at least a portion of the first securing mechanism 70, particularly the portion that would come in direct contact with the patient's skin on the first side 60 of the first strap 50 to provide a comfortable fit when the first strap 50 is secured around the torso 56.

Now referring to FIG. 5, the first strap 50 may also include an attachment mechanism 72, which may be secured to both the first strap 50 and the second strap 52 or it may be secured to just the first strap 50 or just the second strap 52. The attachment mechanism 72 may be configured to be secured with the external coil/transmission coil 24 in a variety of different locations such that when the attachment mechanism 72 is secured to the garment 36, the external coil/transmission coil 24 can be aligned with the internal component/receiving coil 20. The alignment of the transmission coil 24 and the receiving coil 20 can allow the transmission coil 24 to charge the internal receiving coil 20 through, for example, inductive charging, when the transmission coil 24 and the receiving coil 20 are aligned. The receiving coil 20 may be disposed in a variety of different locations within the body and the transmission coil's 24 position would correspond with the location of the receiving coil 20. Also, the external coil/transmission coil 24 may be easily removed from the attachment mechanism 72 to allow the transmission coil 24 to be changed, cleaned, and/or repositioned. The attachment mechanism 72 may include a button or other releasing mechanism to allow for a quick and simple removal of the transmission coil 24.

As shown in FIGS. 4 and 5, the attachment mechanism may be secured to the garment 36 and may have one slot 74 and/or a plurality of slots 74 so that the attachment mechanism 72 may be secured to the first strap 50 and/or the second strap 52 in a variety of different configurations with different anchoring mechanisms. The plurality of slots 74 may be configured to engage with the first strap 50 and/or the second strap 52 so that the attachment mechanism 72 is secured in place while the patient moves in normal day-to-day activities and/or is sleeping. For example, as shown in FIG. 5, the attachment mechanism 72 may have at least two slots 74A and 74B that are engaged with the first strap 50. At least one slot 74A may configured be along an x-axis X of the attachment mechanism 72 as shown in FIG and 4 and one slot 74B on the attachment mechanism 72 that is engaged with the second strap 52 may be along a y-axis Y of the attachment mechanism 72. The slot 74B along the y-axis may be any type of securing mechanism, including a slot that is sown and then attached to a portion of the attachment mechanism 72, so that the second strap 52 can move as the patient moves while the attachment mechanism 72 is still secured in place. The attachment mechanism 72 may also include a semi-rigid mounting plate to help hold the attachment mechanism 72 in place.

Continuing to refer to FIG. 5, the second strap 52 may be configured to fit over or around the shoulder 76 of the patient, as shown in FIGS. 6 and 7. The second strap 52 may include a first portion 78 which may be one segment or more than one segment of the second strap 52. As shown in FIG. 5, the first portion 78 of the second strap 52 may be one segment that may be made out of a highly flexible material, such as elastane or another flexible fabric to allow movement of the second strap 52 with the movement of the shoulder 76. The first portion 78 of the second strap 52 may also be made from material and/or fabric that may stretch at the shoulders allowing movement of the shoulders to be isolated while also controlling tension levels within the garment 36 to minimize the movement of the transmission coil 34. The first portion 78 of the second strap 52 may also be a plastic loop, for example a plastic loop that is approximately 1-5 inches thick. The first portion 78 of the second strap 52 may have a lower stiffness zone to control the tension in the second strap 52 due to movements in the arm and/or shoulder. Certain lower stiffness zones may isolate the transmission coil 24 from other movements while certain higher stiffness zones may assist in anchoring the transmission coil 24 to selected anatomical reference points. It will be understood throughout the application that “high(er) stiffness zone(s)” and/or “high(er) stiffness material(s)” may be at about >about 2× stiffer than the “low(er) stiffness zone(s)” and/or the “low(er) stiffness material(s).” The second strap 52 may generally be made from lower stiffness materials and the first strap 50 may generally be made from higher stiffness materials. This may allow the second strap 52 to provide support without directly coupling alignment to shoulder motion. The second strap 52 may also have a second portion 80 which may be one segment or more than one segment of the second strap 52. As shown in one embodiment in FIG. 5, the second portion 80 may be one segment of the second strap and the second portion 80 may have the same flexibility and/or a different flexibility than the first portion 78 of the second strap 52. For example, the second portion 80 of the second strap 52 may be made from an elastic material that is 1-5 inches thick or another type of material that can move with the patient as the shoulders move and rotate in different directions. The second portion 80 of the second strap 52 may also be made into a rectangular plastic loop that may be approximately 1-5 centimeters thick. The first strap 50 and the second strap 52 may also have thread which is used to secure the first strap 50 and the second strap 52 as well as any components to the first strap 50 and the second strap 52. The first strap 50 may also be reinforced where the first strap 50 is secured to the second strap 52.

In an exemplary embodiment, as shown in FIGS. 4 and 5, the total length of the first strap 50 may be between approximately 10-30 inches with a tolerance of plus or minus 0.5 inches. The first strap 50 may also have a large width, for example, the width of the first strap 50 may be between approximately 1-6 inches to help distribute pressure while still being comfortable without having the user become too warm from the size and thermal insulation from the first strap 50. Additionally, a length as shown as A, of a portion of the first strap 50 as shown in FIG. 4, may be between approximately 5-20 inches with a tolerance of plus or minus 0.5 inches. The width of the first strap may be between approximately 1-3 inches with a tolerance of plus or minus 0.5 inches. Also, a length B of a portion of the second strap 52, as shown in FIG. 5, may be between approximately 5-20 inches as shown between a first slider 68A and a second slider 68B with a tolerance of plus or minus 0.5 inches. The length B may be adjusted to different lengths depending upon the placement of the sliders 68 on the second strap 52. It will be understood that each of the lengths provided are exemplary lengths and can be adjusted to a variety of different lengths to accommodate different sizes and shapes. The second slider 68B may allow for vertical size adjustments and can be adjusted on or off to the body of the patient. A second length, shown as C in FIG. 5, of a portion of the second strap 52 may be between approximately 6-20 inches with a tolerance of plus or minus 0.5 inches. A length D (not fully shown) of a portion of the first strap 50 between the attachment mechanism 72 and the first material segment 66 where the first material segment 66 is secured to the first securing mechanism 70 may be between approximately 5-20 inches with a tolerance of plus or minus 0.5 inches. The second strap 52 may have a minimum length, shown as E in FIG. 5, of between approximately 15-30 inches with a tolerance of plus or minus 0.5 inches. The length of the second strap 52 between the slider 68B and the slot 74A, as shown in FIG. 5, 26 may be between approximately 5-15 inches with a tolerance of plus or minus 0.5 inches.

Now referring to FIGS. 6 and 7, the exemplary embodiment of the alignment garment is shown being worn on the torso 56 of the patient. FIG. 6 is a front view of a patient wearing the exemplary garment 36 as shown in FIGS. 4 and 5 and FIG. 7 is a rear view of the patient wearing the same exemplary garment 36. The first strap 50 may be around the lower bust portion of the torso 56, for example, the first strap 50 may be around the base of the sternum so that the first strap 50 is clear of the breast and/or pectoral region, of the patient and help to anchor the garment 36 to the patient. Additionally, the attachment mechanism 72 may sit in on the side of the patient, including but not limited to the axilla region of the patient's body, depending upon where the receiving coil 20 is located within the patient's body. The second strap 52 can be made smaller and larger such that the attachment mechanism 72 can be moved to different portions of the patient's body. The attachment mechanism 72 may configured to engage with the transmission coil 24 and help to maintain alignment between the transmission coil 24 and the receiving coil 20 so that that receiving coil 20 can be inductively powered through the patient's skin by the transmission coil 24 (as seen in FIG. 2) while the garment 36 maintains the alignment of these two components while the patient moves. The attachment mechanism 72 may be movable so that it can be secured in a variety of different locations in the body to allow for charging to occur on the internal and external components.

Continuing to refer to FIGS. 6-7, the patient's torso 56 may define a longitudinal axis Y₂ and the first portion 78 of the second strap 52 may be movable in a plane that is at least substantially orthogonal to the longitudinal axis Y₂ defined by the patient's torso 56. This type of movement can provide stability and limit movement when the second strap 52 is secured to the attachment mechanism 72. The placement of the first strap 50 and the second strap 52 concentrates tension through the middle of the receiving coil 24 that is secured within the attachment mechanism 72 so that the receiving coil 24 and the attachment mechanism 72 may deform to the shape of the particular person's body. The first strap 50 may be secured with the second strap 52 in the middle of the patient's back for stability. In one embodiment, the second strap 52 may be secured to the first material segment 66 on the first strap 50. The first strap 50 can provide horizontal stability to the receiving coil 24 and the attachment mechanism 72 while the second strap 52 can provide vertical stability to the receiving coil 24 and the attachment mechanism 72. The receiving coil 24 may be in close contact with the skin with the first strap 50 and the receiving coil 24 may include a friction contact material or fabric such as a silicon material to maintain contact with the skin. The various components of the garment 36 may be slim-lined as much as possible to prevent bulkiness of the garment 36 when being worn.

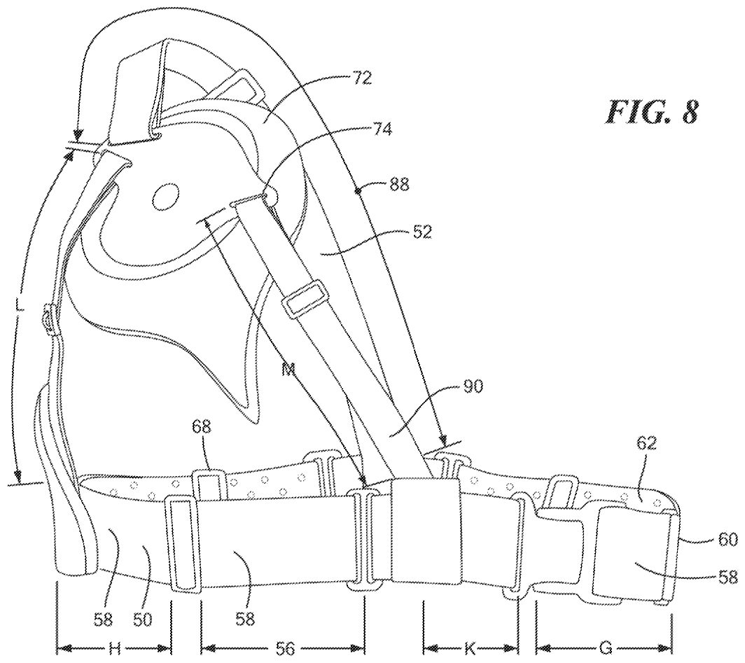

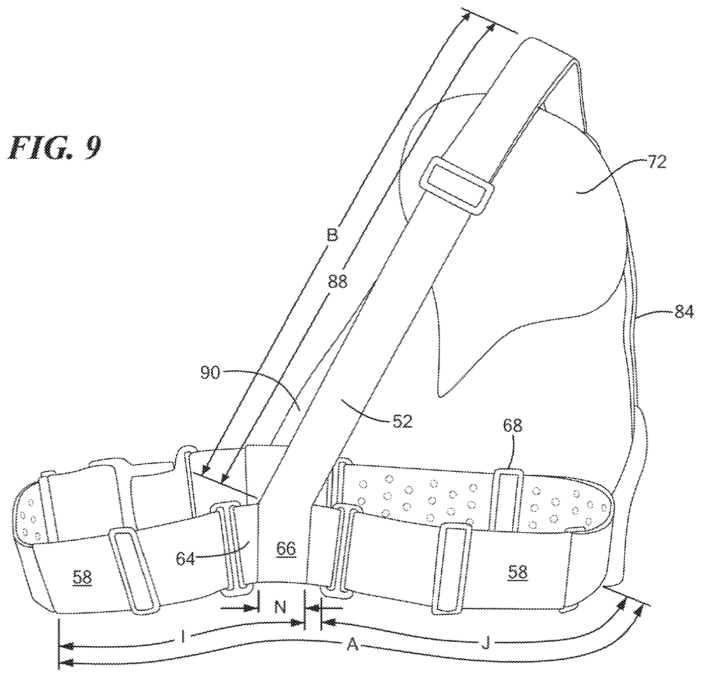

Now referring to FIGS. 8-9, there is shown another exemplary configuration of the garment 36. In this exemplary garment 36, the attachment mechanism 72 may be positioned to engage with the external charging component, the transmission coil 24, where the internal charging component, the receiving coil 20, is located in the patient's pectoral region 82.

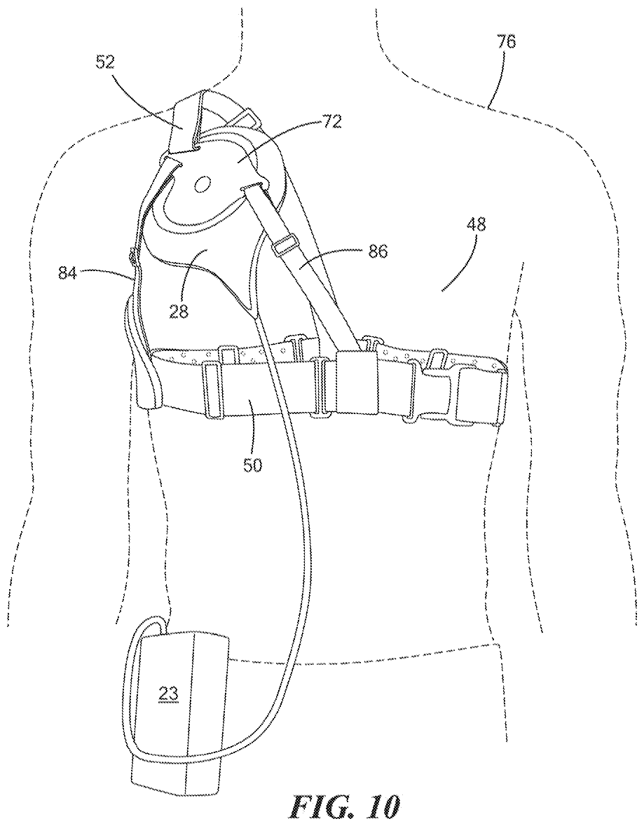

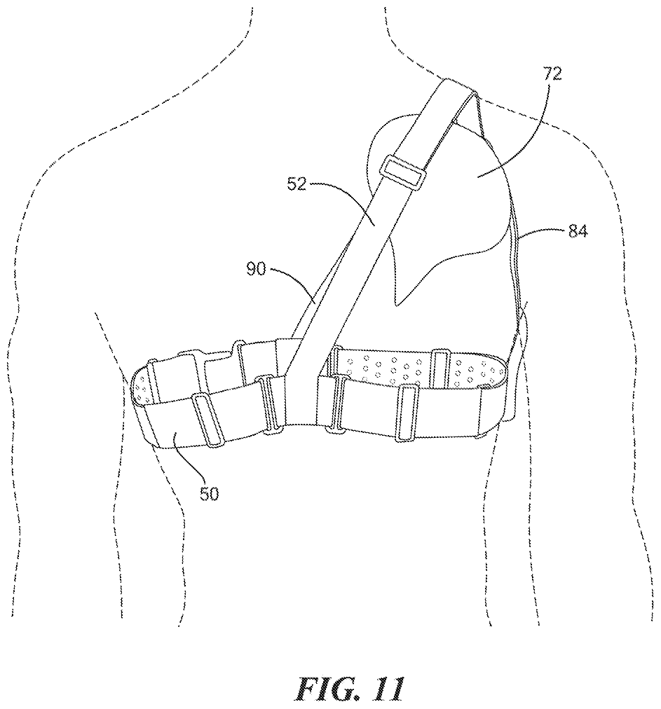

In FIGS. 8-11, the garment 36 may include the first strap 50, the second strap 52, a third strap 84, and a fourth strap 86. In one configuration, the first strap 50 may be fitted to secure around the torso 56 of the patient just below the sternum, the bust and the pectoral region, as shown in FIGS. 10 and 11. The first strap 50 may be made from flexible material so that the first strap 50 can be configured to fit different body shapes and sizes. In one configuration, the first strap 50 may include the first portion 58 which may be one segment in the first strap 50 or more than one segment as shown in FIGS. 8 and 9. For example, in one exemplary embodiment of the garment 36, the first strap 50 may have seven first portion 58 segments that may be made from the same material or different materials. It will be understood that there may be more or fewer segments in the first portion 58 that make up the first strap 50 and the determination of how many segments to include may be based upon the patient's body type as well as comfort considerations for the patient. The first strap 50 may be made from the flexible and stretchy material including a plush elastic to make the first strap 50 fit around different sized and shaped torsos 56. The plush elastic may have a foundation with elastic with one soft and velvety side which is on the side of the first strap 50 that is in contact with the skin of the patient. For example, the first strap 50 may have the first side 60 and the second side 62 where the first side 60 contours around the torso 56 of the patient and may be in direct contact with the patient's skin. Additional material or different material may be integrated into the first side 60 to make the garment 36 more comfortable to wear.

Continuing to refer to FIGS. 8 and 9, the first strap 50 may also include the second portion 64 which may be made from material with stretchy and elastic qualities while also being able to stay in place on the patient's body. For example, the second portion 64 of the first strap 50 may be made out material which is stretchy on the second side 62 and has a material on the first side 60 that is designed to keep the second portion 64 of the first strap 50 in place on the patient's skin. For example, the second portion 64 may use dot silicon elastic which is stretchy on the second side 62 and has silicon dots on the first side 60 to help keep the first strap 50 in place.

In FIGS. 8 and 9, the first strap 50 may also include the first material segment 66 and the first material segment 66 may be configured so that the first strap 50 may comfortably fit around a portion of the torso 56 of the patient. The first material segment 66 may be made from a fabric that be placed in direct contact with a patient's skin and cause minimal irritation including fabrics like polyester, viscose, rayon, and spandex and/or a combination of these materials. The material may be a double knit jersey dress fabric. In one example, as shown in FIG. 9, the first material segment 66 may be cut into the shape of a right triangle to help cover the right angle between the first strap 50 and the third strap 84 which can prevent rubbing against the skin of the patient and make the garment 36 more comfortable to wear for long periods of time as the patient moves throughout the day and night.

Continuing to refer to FIGS. 8 and 9, the second strap 52 may have a second material segment 88 and the second material segment 88 may be made from elastic material that can move easily and comfortably with the patient. The second strap 52 may be configured to fit over the shoulder 76 of the patient to help secure the garment 36 in place and as a non-limiting example may be made out of an elastic strap that is between approximately 1-5 inches in width to allow for patient movement while also securing the second strap 52 in place. The second strap 52 may also be made from material and/or fabric that may stretch at the shoulders allowing movement of the shoulders to be isolated while also controlling tension levels within the garment 36 to minimize the movement of the transmission coil 34. The first strap 50, the second strap 52, the third strap 84, and the fourth strap 86 may also include at least one slider 68 or more than one slider 68. In an alternate configuration, there may not be any sliders 68 as part of the straps and each of the straps may be custom fitted to the patient such that sliders 68 are not necessary to size the strap for the patient. The number of sliders 68 on the first strap 50, the second strap 52, the third strap 84, and the fourth strap 86 may be customized for the patient so that each strap can easily and comfortably fit the patient. For example the slider 68 may be between approximately a 1-5 inch plastic slider, between approximately a 1-5 inch plastic slider/loop, or any other size and shape slider depending upon the size, shape, and thickness of the first strap 50, the second strap 52, the third strap 84, and the fourth strap 86 as well as how each of the straps may need to be adjusted for the patient. Each slider 68 may be made out of any material to make it comfortable for the patient to wear for prolonged periods of time and any portion of the slider 68 that is in direct contact with the patient's skin may be covered with a material to make it comfortable to wear for long periods of time.

Continuing to refer to FIG. 8, the first strap 50 may also include the first securing mechanism 70 to make it easier to secure the first strap 50 around the torso 56. In one embodiment, the first securing mechanism 70 may be a buckle such as a frog clip or another type of releasable clip as well as a G-hook to help secure the first strap 50 in place. Also, the first material segment 66 may be secured to the first securing mechanism 70 on the first side 60 of the first strap 50 so that the first securing mechanism 70 is not in direct contact with the patient's skin and the first material segment 66 directly contacts the skin. The material segment 66 may be a fabric tab sitting behind the first securing mechanism 70 to create a soft feel against the skin and prevent the first securing mechanism 70 from digging into the skin.

Now referring to FIGS. 8-9, the second strap 52, the third strap 84, and the fourth strap 86 may also include the attachment mechanism 72, and the attachment mechanism 72 may be secured by the second strap 52, the third strap 84, and the fourth strap 86. In the non-limiting example, as shown in FIGS. 8-11, the attachment mechanism 72 may include the plurality of slots 74 so that the attachment mechanism 72 may be secured to the second strap 52, the third strap 84, and the fourth strap 86 with at least one slot 74 configured to be secured through each of the straps. The plurality of slots 74 may be configured to engage with the second strap 52, the third strap 84, and/or the fourth strap 86 to limit the movement of the attachment mechanism 72 when the patient moves and may secure the attachment mechanism 72 in place in the pectoral region 82 as seen in FIGS. 10-11. For example, as shown in FIGS. 8 and 9, the attachment mechanism 72 may have the plurality of slots 74 including one slot 74 that is engageable with the second strap 52, one slot 74 that is engageable with the third strap 84 and one slot 74 that is engageable with the fourth strap 86. This type of configuration can allow for movement of the patient while maintaining the attachment mechanism 72 in place in the pectoral region 82.

Continuing to refer to FIG. 10, the second strap 52 may be configured to fit over the shoulder 76 of the patient, as shown in FIGS. 10 and 11. The third strap 84 may be configured to be secured between the first strap 50 and the attachment mechanism 72 such that the third strap 84 is near the patient's right side and sits below the attachment mechanism 72 and above the first strap 50. A fourth strap 86 may have a third material segment 90 and the third material segment 90 may be made from elastic material. In one example the elastic may be made from elastic material with a length of between approximately 0.5-3.0 inches to allow for movement of the fourth strap 86. The fourth strap 86 may also be narrow to fit between the breasts of the patient. In one embodiment, the width of the fourth strap 86 may be approximately between 0.1 inches-1.5 inches. The fourth strap 86 may be connected to the first strap 50 with the first material segment 66 and connected to the attachment mechanism 72 such that the fourth strap 86 is configured to sit near the patient's pectoral region 82 as shown in FIGS. 10-11. This type of configuration may be used to fit around the breast of the patient comfortably.

In an exemplary embodiment as shown in FIGS. 8-11, the total circumference of the first strap 50, shown as A in FIG. 9, may be between approximately 20-60 inches with a tolerance of plus or minus 0.5 inches. Additionally, in an exemplary embodiment, a length G of a portion of the first strap 50 between the first securing mechanism 70 and the first portion 58 of the first strap 50, as shown in FIG. 8, may be approximately between 2-10 inches with a tolerance of plus or minus 0.5 inches. A length of the second portion 64 of the first strap 50, as shown in FIG. 9, may be approximately between 2-10 inches with a tolerance of plus or minus 0.5 inches. A length of the first portion 58 between two sliders 68, as shown as a length H in FIG. 8, may be approximately between 2-10 inches in length with a tolerance of plus or minus 0.5 inches. A length of the first strap 50 between one slider 68 and a portion of the first material segment 66, shown as length I in FIG. 9, may be approximately 5-20 inches with a tolerance of plus or minus 0.5 inches. A length of the first strap 50 between a portion of the first material segment 66, and where the third strap 84 is joined to the first strap 50, shown as a length J in FIG. 9, may be approximately between 2-15 inches with a tolerance of plus or minus 0.5 inches. A length of the third strap 84 between the attachment mechanism 72 and where the third strap 84 is joined to the first strap 50, shown as a length L in FIG. 8, may be approximately between 1-15 inches with a tolerance of plus or minus 0.5 inches. A length of the fourth strap 86 between the attachment mechanism 72 and where the third strap 84 is joined to the first strap 50, shown as a length M in FIG. 8, may be approximately between 1-15 inches with a tolerance of plus or minus 0.5 inches. The length of the second strap 52, as shown as a length B in FIG. 9, may be approximately between 5-40 inches with a tolerance of plus or minus 0.5 inches. A length of the first material segment 66, as shown as a length N in FIG. 9, may be approximately between 3-20 inches with a tolerance of plus or minus 0.5 inches.

Now referring to FIGS. 10-11, the exemplary embodiment of the garment 36 as shown being worn on the torso 56 of the patient. The garment 36 may be the strap based system that uses a series of elastic straps to maintain the alignment of the attachment mechanism 72 with the patient's pectoral region 82. The first strap 50 may be like a belt, while the second strap 52, the third strap 84, and the fourth strap 86 may be alignment straps for the attachment mechanism 72 to secure the attachment mechanism 72 to the pectoral region 82. The second strap 52, the third strap 84 and the fourth strap 86 may be secured to the attachment mechanism 72 and connect the attachment mechanism 72 to the first strap 50 to create a vertical tension loop and provide horizontal stability. The first strap 50 may have a slider 68 or some other type of adjustment mechanism on the first strap 50 in the area between where the third strap 84 and the fourth strap 86 are secured to the first strap 50 so that the first strap 50 can be readily adjustable to accommodate different breast sizes and so that the straps do not impinge on breast tissue. The stiffness of the straps 50, 52, 84, and 86 may be adjustable to adjust the tension of the garment 36 while the body moves into different positions. The angles of the second strap 52, the third strap 84 and the fourth strap 86 may be adjustable to provide stability to the attachment mechanism 72 and the transmission coil 24 when the transmission coil 24 is secured to the attachment mechanism 72 form multiple directions and various movements of the body.

FIG. 10 is a front view of a patient wearing the garment 36 as shown in FIGS. 8-9 and FIG. 11 is a rear view of the patient wearing the same garment 36. The first strap 50 may be around the lower bust of the patient and anchor the garment 36 to the patient. The third strap 84 and the fourth strap 86 may be configured to sit on either side of the right breast as shown in FIG. 10. The sliders 68 on the first strap 50 may allow the angles of the third strap 84 and the fourth strap 84 to be adjusted to fit in a comfortable position around the breasts and underarm area. Additionally, the attachment mechanism 72 may sit in the pectoral region 82 of the patient's body, depending upon where the receiving coil 20 is located within a patient's body and how the transmission coil 24 needs to be configured in order to transmit energy between the transmission coil 24 and the receiving coil 20. The second strap 52 can be made smaller and larger such that the attachment mechanism 72 can be moved to different portions of the patient's body. Additionally, the third strap 84 and the fourth strap 86 may have sliders 68 which can control and move the position of the attachment mechanism 72 relative to the first strap 50 while also controlling the tension on the third strap 84 and the fourth strap 86. The attachment mechanism 72 may configured to engage with the transmission coil 24 and help to maintain alignment between the transmission coil 24 and the receiving coil 20 can be inductively powered through the patient's skin by a transmission coil 24 (as seen in FIG. 2) while the garment 36 maintains the alignment of these two components while the patient moves. The third strap 84 and the fourth strap 86 may aid in positioning the attachment mechanism 72 over the pectoral region 82. The second strap 52 may create a vertical tension loop with the first strap 50, the third strap 84, and the fourth strap 86 to hold the attachment mechanism 72 and the receiving coil 24 which is secured by the attachment mechanism 72 against the skin. The tension in the first strap 50 may be adjustable so that it is comfortable as the shoulder moves. As a non-limiting example, the slider 68 may be adjusted to change the tension in the first strap 50.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

1. An alignment garment for holding an external coil in a predetermined location, the alignment garment comprising:
an attachment mechanism for holding the external coil in the predetermined location; and
a plurality of straps including a first strap and a second strap each being secured to the attachment mechanism.

2. The alignment garment of claim 1, wherein each strap in the plurality of straps is adjustable to different lengths.

3. The alignment garment of claim 1, wherein the first strap of the plurality of straps has a first portion and a second portion, the first portion being configured to be secured to the attachment mechanism and the second portion being secured to the second strap of the plurality of straps.

4. The alignment garment of claim 3, wherein the first strap of the plurality of straps has a length of 10 inches to 30 inches.

5. The alignment garment of claim 3, wherein the second portion of the first strap of the plurality of straps is made from a material that has a lower elasticity than the first portion to limit movement in the second portion of the first strap.

6. The alignment garment of claim 3, wherein the first portion of the first strap of the plurality of straps is moveable along a single axis.

7. The alignment garment of claim 3, wherein the second strap of the plurality of straps has a first portion and a second portion, the first portion and the second portion of the second strap being configured to be secured to the attachment mechanism.

8. The alignment garment of claim 1, wherein each strap of the plurality of straps includes at least one slider, the at least one slider being configured to adjust the respective strap of the plurality of straps to different lengths.

9. The alignment garment of claim 8, wherein the at least one slider is made from a non-conductive material.

10. An alignment garment for holding an external coil in a predetermined location, the alignment garment comprising:
an attachment mechanism for holding the external coil in the predetermined location;
a first strap having a circumference of 20 inches to 60 inches;
a second strap secured to the attachment mechanism and the first strap, the second strap having a length of 15 inches to 40 inches;
a third strap secured to the attachment mechanism and secured to the first strap; and
a fourth strap secured to the attachment mechanism and the first strap.

11. The alignment garment of claim 10, wherein the third strap has a length of 5 inches to 15 inches.

12. The alignment garment of claim 10, wherein the fourth strap has a length of 5 inches to 15 inches.

13. The alignment garment of claim 10, wherein the first strap has a first slider proximate where the third strap is secured to the first strap and the first strap has a second slider proximate where the fourth strap is secured to the first strap.

14. The alignment garment of claim 10, wherein the first strap has at least three sliders configured to adjust a length of the first strap.

15. An alignment garment for holding an external coil in a predetermined location, the alignment garment comprising:
an attachment mechanism for holding the external coil in the predetermined location;
a first strap having a circumference of 20 inches to 60 inches;
a second strap secured to the attachment mechanism and the first strap, the second strap having a length of 15 inches to 40 inches;
a third strap secured to the attachment mechanism and secured to the first strap, the third strap having a length of 5 inches to 15 inches; and
a fourth strap secured to the attachment mechanism and the first strap, the fourth strap having a length of 5 inches to 15 inches.

16. The alignment garment of claim 15, wherein the first strap includes a first slider proximate where the third strap is secured to the first strap and the first strap includes a second slider proximate where the fourth strap is secured to the first strap.

17. The alignment garment of claim 15, wherein the second strap is made from a material that has a lower elasticity than the first strap to limit movement of the second strap.

18. The alignment garment of claim 10, wherein the second strap is made from a material that has a lower elasticity than the first strap to limit movement of the second strap.

19. The alignment garment of claim 10, wherein the first strap includes a securing mechanism configured to releasably secure the first strap.

20. The alignment garment of claim 10, wherein the first strap includes a reinforcing mechanism disposed where the first strap is secured to the second strap.
